(19) United States
(12) Patent Application Publication
(10) Pub. No.: US 2015/0192560 A1
(43) Pub. Date: Jul. 9, 2015

(54) METHOD OF ASSESSING OIL CONDITION IN AN ENGINE

(71) Applicant: Caterpillar Inc., Peoria, IL (US)

(72) Inventors: Amiyo K. Basu, Peoria, IL (US); Anthony Petrou, Peoria, IL (US)

(73) Assignee: Caterpillar Inc., Peoria, IL (US)

(21) Appl. No.: 14/149,050

(22) Filed: Jan. 7, 2014

(51) Int. Cl. G01N 33/28

(52) U.S. Cl. CPC G01N 33/2888

(57) ABSTRACT

A method of assessing oil condition in an engine includes receiving an engine variable including at least one of: a type of the engine, a displacement of the engine, a property of fuel used in the engine, and a property of oil used in the engine. The method further includes determining an operating parameter of the engine including at least one of an oil temperature, an oil pressure, a duty-cycle of the engine, a load on the engine, a speed of the engine, a power output of the engine, a rate of fuel consumption by the engine, and a time period elapsed since last change of oil in the engine. The method further includes calculating a rate of degradation of oil based on the determined engine variable and the determined operating parameter. The method further includes calculating a remaining service-life of the oil from the calculated rate of degradation.

### TECHNICAL FIELD

The present disclosure relates to engine oil, and more particularly to a method of assessing oil condition in an engine.

### BACKGROUND

Typically, replacement of oil in an engine is scheduled based on factors such as engine mileage, or time elapsed from last change of oil in the engine. However, these factors may be insufficient to determine accurately the current condition or quality of oil, and therefore a remaining service life of the oil.

Many methods have been contemplated in the past for determining the current condition or quality of oil in engines. However, some methods may be difficult to implement and integrate with existing systems of the engine such as, for example, an onboard computer, or an electronic control module (ECM). Other methods may require inclusion of additional components such as sensors, gauges, transducers, and other components, thereby increasing complexity and costs associated with the system. Moreover, implementation of previously known methods may render the determination of oil condition or quality inaccurate.

### SUMMARY OF THE DISCLOSURE

In one aspect, the present disclosure discloses a method of assessing oil condition in an engine. The method includes receiving an engine variable including at least one of a type of the engine, a displacement of the engine, a property of fuel used in the engine, and a property of oil used in the engine. The method further includes determining an operating parameter of the engine including at least one of an oil temperature, an oil pressure, a duty-cycle of the engine, a load on the engine, a rotational speed of the engine, a power output of the engine, a rate of fuel consumption by the engine, and a time period elapsed since last change of oil in the engine. The method further includes calculating a rate of degradation of oil based on the determined engine variable and the determined operating parameter. The method further includes determining a remaining service-life of the oil from the calculated rate of degradation.

Other features and aspects of this disclosure will be apparent from the following description and the accompanying drawings.

### BRIEF DESCRIPTION OF THE DRAWINGS

FIG. 1 is a schematic illustration of an exemplary system employing a method of the present disclosure;

FIG. 2 is the method for carrying out assessment of oil condition in an engine, in accordance with an embodiment of the present disclosure;

FIGS. 3-4 are exemplary graphical representations plotting rate of fuel consumption of the engine in a duty cycle versus total operation hours in the duty-cycle of the engine, in accordance with an embodiment of the present disclosure;

FIGS. 5-6 are exemplary plots of a rotational speed of the engine in a duty cycle versus total operation hours in the duty-cycle of the engine;

FIG. 7 is an exemplary two-dimensional map of multiplier values based on oil temperature;

FIG. 8 is an exemplary three-dimensional map of multiplier values based on rate of fuel consumption of the engine and the rotational speed of the engine; and

FIGS. 9-10 are exemplary graphical representations showing degradation in oil condition measured by employing the method of the present disclosure.

### DETAILED DESCRIPTION

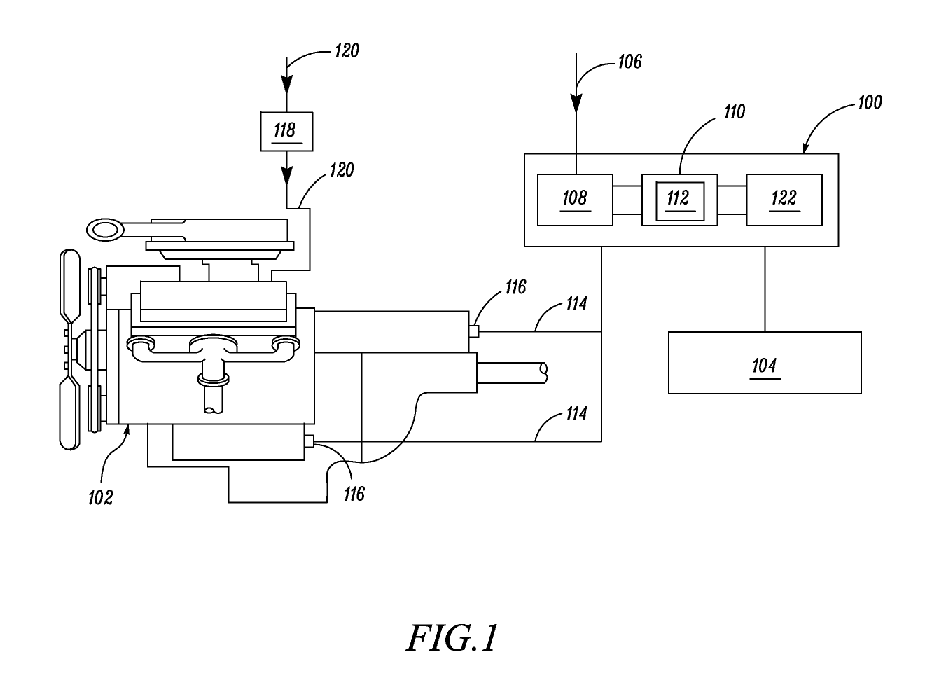

The present disclosure relates to engine oil, and more particularly to a method of assessing oil condition in an engine. FIG. 1 shows a schematic illustration of an exemplary system 100 employing the method of the present disclosure. The system 100 is configured to assess oil condition in the engine 102. The engine 102 may include any type of engine known in the art such as, but not limited to, a diesel engine, a gasoline engine, or a dual-fuel engine.

The engine 102 may be coupled with a transmission system (not shown) to drive a piece of equipment (not shown), such as, for example, a generator, a compressor, or a pump. The transmission system, disclosed herein, may include any type of transmission system commonly known in the art such as, for example, hydrostatic transmissions, hydrodynamic transmissions, electric transmissions, Continuously Variable Transmissions (CVT), or Electric Variable Transmission (EVT).

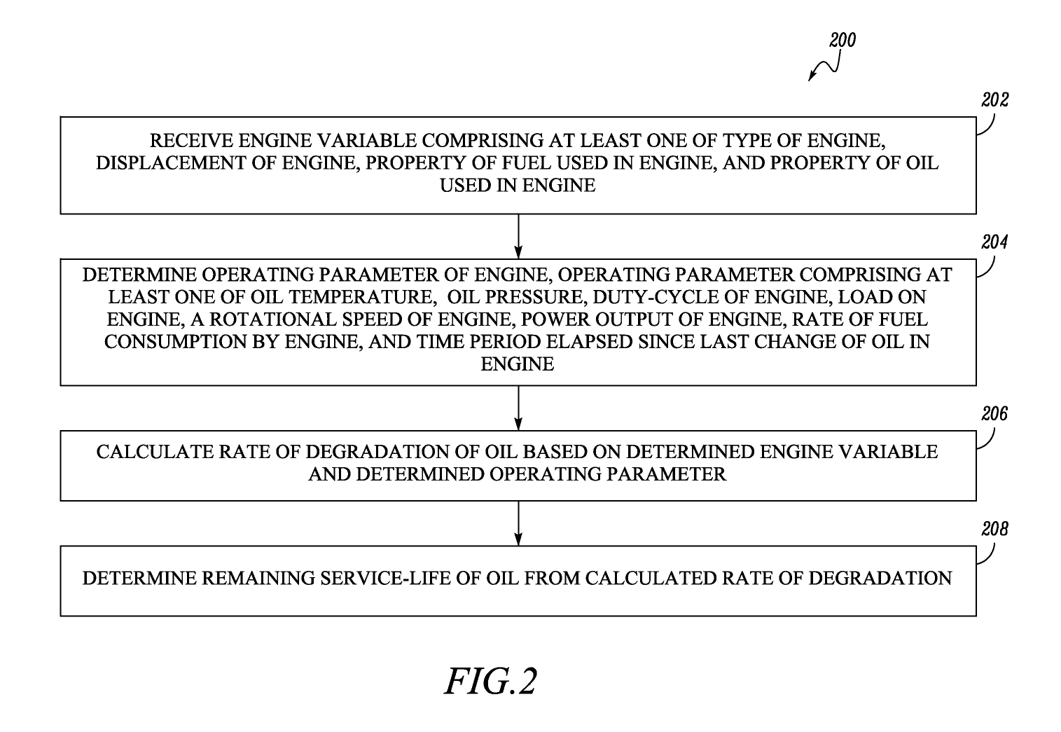

The system 100 is further configured to command a display device 104 for displaying the assessed condition of oil. The system 100 may provide one or more output signals for displaying the assessed condition of oil at the display device 104. Some devices for presenting data indicative of the assessed condition of oil may include, for example, an onboard computer or a hand-held electronic device. The formats of data presentation may include graphs, charts, or percentages, but are not limited thereto. Information on devices and formats for presenting data is readily available to those skilled in the art and hence, explanation of the same is omitted herein. Steps pertaining to the assessment of oil condition by the system 100 will be discussed hereinafter in conjunction with FIGS. 2-10.

FIG. 2 illustrates a method 200 of assessing the oil condition in the engine 102. At step 202, the method 200 includes receiving an engine variable 106. The engine variable 106, disclosed herein, includes at least one of a type of the engine, a displacement of the engine, a property of fuel used in the engine, and a property of oil used in the engine. Referring to FIG. 1, in an embodiment, the engine variable 106 may be input to the system 100 by an operator of the engine 102. The system 100 disclosed herein, may include an input device 108, for example, a selector switch, a keypad with physical buttons, a touchpad, or any Graphical User Interfaces (GUI) commonly known in the art. The input device 108 facilitates the operator to provide the engine variable 106 to the system 100. The system 100 may additionally include a memory unit 110 having one or more repositories 112 therein. The repositories 112 may include, but not limited to, models or types of engines, type of fuel, type of oil, and displacement of the engine amongst other engine variables 106 that may facilitate the operator to select the appropriate engine variable 106 directly therefrom. For example, the operator may provide the system 100 with inputs indicative of the engine variables 106, such as, a 6.0-liter diesel engine employing oil of viscosity index 80.

At step 204, the method 200 includes determining an operating parameter 114 of the engine 102. The operating parameter 114, disclosed herein, includes at least one of an oil temperature, an oil pressure, a duty-cycle of the engine, a load on the engine, a rotational speed of the engine, a power output of the engine, a rate of fuel consumption by the engine, and a time period elapsed since last change of oil in the engine. The system 100 may be provided with one or more sensors 116 associated with various components of the engine 102. The sensors 116 may be configured to sense the operating parameters 114 and provide the system 100 with signals representative of the operating parameters 114. The sensors 116 may include, but not limited to, speed sensors, pressure sensors, thermocouple junctions, or other transducers to obtain and provide the operating parameters 114 to the system 100. In an event where the sensors 116 provide analog signals, the system 100 may additionally include an analog-to-digital converter (A/D) (not shown) for converting the analog signals into digital form.

The operating parameters 114 may be measured by sensors 116 located at numerous locations or points of the engine 102. For example, the oil temperature may be obtained from a temperature sensor located in a coolant circuit (not shown) of the engine 102. Alternatively, the oil temperature may be obtained from a temperature sensor located at an exhaust port (not shown) of the engine 102. The temperature sensors provide a direct temperature of the coolant in the coolant circuit or a temperature of the exhaust at the exhaust port. Thereafter, the oil temperature may be mathematically deduced from the temperatures of coolant and/or exhaust by using one or more equations that are commonly known to a person ordinarily skilled in the art.

In an embodiment, the oil temperature may be measured from a measured rate of fuel consumption by the engine 102. For example, the system 100 may be additionally provided with a fuel-metering device 118 located in a fuel input line 120 of the engine 102, and the oil temperature may be deduced mathematically by employing various commonly known equations after receiving a signal indicative of the fuel consumption from the fuel-metering device 118.

It may be noted that the system 100, disclosed herein, may additionally include processing components 122 such as, for example, a processor, a microprocessor, or a general-purpose computer to perform co-relations and deduce various operating parameters 114 therefrom (See FIG. 1). Further, the memory unit 110 may be configured to include experimental test data, for example, pre-calculated tables, curves, or graphs that are obtained from various theoretical models, statistical models, simulated models, or combinations thereof. Accordingly, the processing components 122 of the system 100 may be configured to look-up the memory unit 110 for deducing the operating parameters 114 from the experimental test data.

In another embodiment, the sensors 116, for example, the pressure transducers, as disclosed earlier herein, may be configured to measure the oil pressure. The pressure transducers may be positioned at various locations of the engine 102 such as, for example, an oil gallery, an oil pump, or a crankcase associated with the engine 102.

The system 100 is additionally provided with signals representative of duty-cycles of the engine 102, and rotational speed of the engine 102. The duty-cycles and the rotational speed of the engine 102 may be expressed in terms of, but not limited to, hours of engine operation and revolutions per minute, respectively. Further, the system 100 may be additionally provided with one or more counters (not shown) to count intermittent operation hours in the duty-cycles and allow resetting of the duty-cycles to an initial value, for example, zero operation hours when oil is changed in the engine 102. Additionally, the counters may be configured to measure the rotational speed of the engine 102 and may include components commonly known in the art, such as, but not limited to, a rotary encoder.

In an embodiment, the system 100 may be configured to determine load on the engine 102. The fuel-metering device 118 may be further configured to provide data pertaining to load on the engine 102. As is the case with oil temperature, the load on the engine 102 may be deduced mathematically by employing various commonly known equations after receiving a signal indicative of the rate of fuel consumption from the fuel-metering device 118. The memory unit 110 may be pre-stored with experimental test data pertaining to correlation between the rate of fuel consumption and the load on the engine 102. This experimental test data may be obtained from various theoretical models, statistical models, simulated models, or combinations thereof, such that the processing components 122 may look-up the memory unit 110 for deducing the load on the engine 102 from the experimental test data.

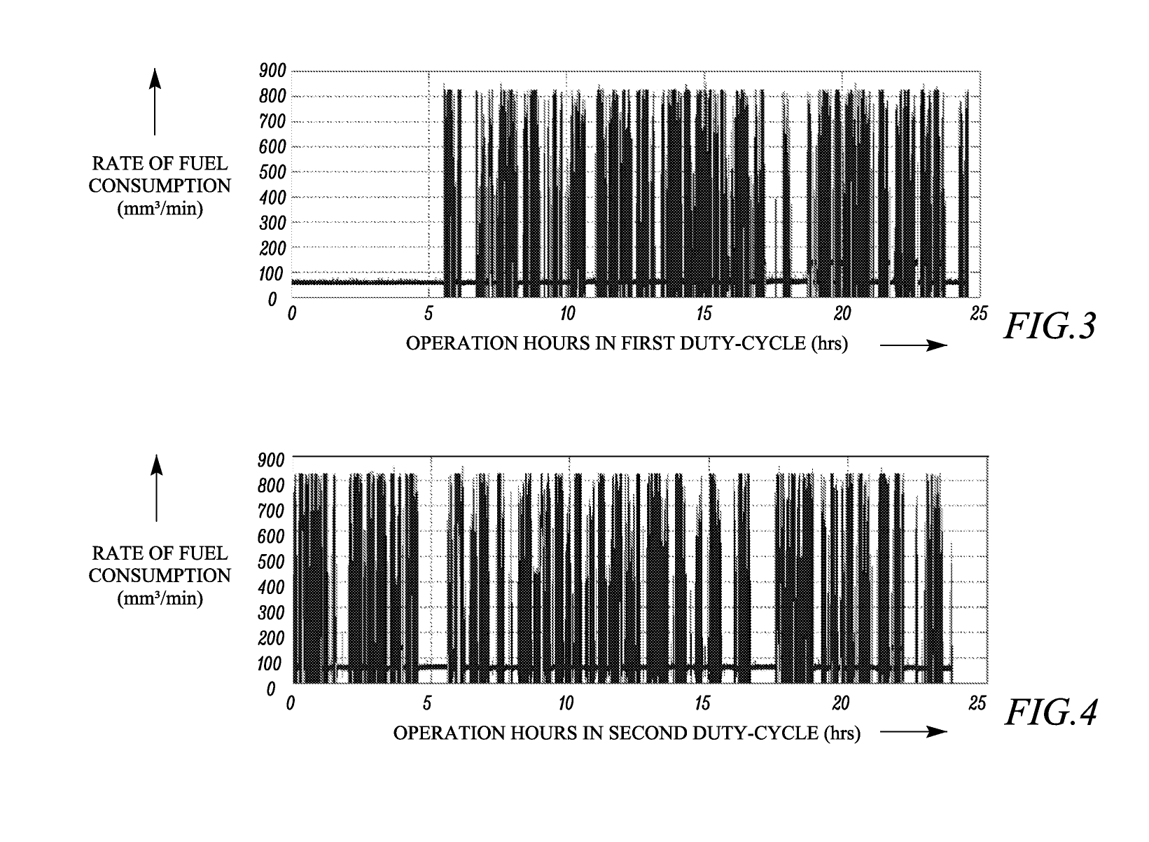

Referring again to FIG. 2, at step 206, the method 200 further includes calculating a rate of degradation of oil based on the determined engine variable 106 and the determined operating parameter 114. Now referring to FIGS. 3-4, exemplary plots of rate of fuel consumption of the engine 102 versus total operation hours in a duty-cycle of the engine 102 are provided. Specifically, FIG. 3 is a plot obtained from a first duty-cycle of the engine 102, for example, on a first day of operation of the engine 102, while FIG. 4 is a plot obtained from a second duty-cycle of the engine 102, for example, on a second day of operation of the engine 102. It can be seen that the first duty-cycle and the second duty cycle present different rates of fuel consumption.

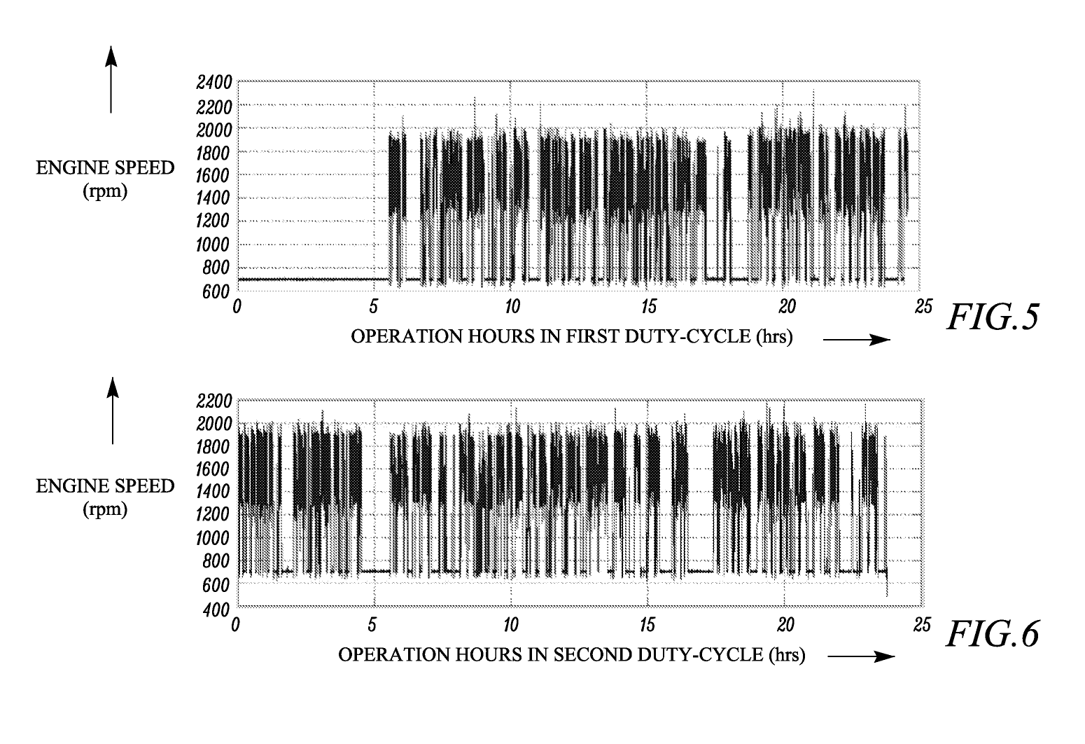

Similarly, referring to FIGS. 5-6, exemplary plots of the rotational speed of the engine 102 in a duty cycle versus total operation hours in the duty-cycle of the engine 102 are provided. Specifically, FIG. 5 is a plot obtained from the first duty-cycle of the engine 102, for example, on the first day of operation of the engine 102, while FIG. 6 is a plot obtained from the second duty-cycle of the engine 102, for example, on the second day of operation of the engine 102. It can be seen that the first duty-cycle and the second duty cycle present different of rotational speed of the engine 102.

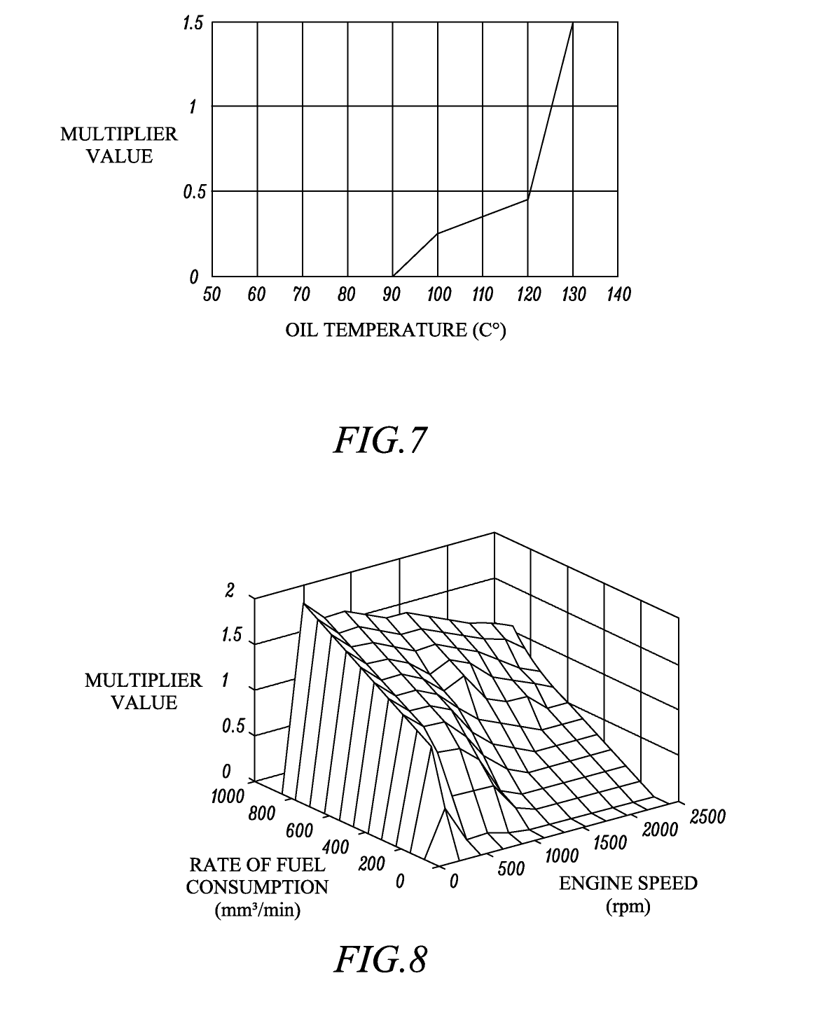

Referring to FIG. 7, the system 100 of the present disclosure is configured to generate a two-dimensional map of multiplier values based on oil temperature. For example, based on the received engine variables 106 and the determined operating parameters 114, the processing components 122 may generate multiplier values to be used in calculating the rate of the rate of degradation of oil. For example, as shown in FIG. 7, if the oil temperature is 123 degree centigrade, a multiplier value of 0.5 may be employed by the processing components 122 while calculating the rate of degradation of oil. In another example, if the oil temperature continues to increase and is 126 degree centigrade, a multiplier value of 1.0 may be employed by the processing components 122 while calculating the rate of degradation of oil.

In one embodiment, the processing components 122 alone may determine the various threshold temperature values and apply the appropriate multiplier values. Alternatively, the repositories 112 in the memory unit 110 may be additionally configured to store such threshold temperature values based on experimental test data. Accordingly, the processing components 122 of the system 100 may look-up the memory unit 110 for obtaining an appropriate multiplier value and calculating the rate of degradation of oil. It is to be noted that the experimental test data, disclosed herein, may be data determined during trial runs of the engine 102, or may include actual data from prior duty-cycles of the engine 102, or data determined by calculations performed at a design stage of the engine 102.

FIG. 8 depicts an exemplary three-dimensional map of multiplier values based on the rate of fuel consumption of the engine 102 and the rotational speed of the engine 102, according to an embodiment of the present disclosure. As with the case of the two-dimensional map shown in FIG. 7, the system 100 of the present disclosure is configured to generate three-dimensional maps of multiplier values based on the operating parameters 114. However, in the three-dimensional map of FIG. 8, at least two operating parameters 114 have been simultaneously considered to determine the multiplier values. Although the map of FIG. 7 is plotted based on oil temperature, and the map of FIG. 8 is plotted based on the rate of fuel consumption and rotational speed of the engine 102, it must be noted that any of the operating parameter(s) 114 of the present disclosure may be used to plot the two-dimensional or three-dimensional maps, respectively. Alternatively, the memory unit 110 of the present disclosure may be additionally configured to store such maps in the repository 112 based on experimental test data. Accordingly, the processing components 122 of the system 100 may look-up the memory unit 110 for obtaining an appropriate multiplier value and calculating the rate of degradation of oil. Therefore, these maps help the system 100 in accurately calculating the rate of degradation of oil by taking into account correlations of one or more operating parameters 114 via the two-dimensional and three-dimensional maps.

For example, referring to FIG. 8, if the rate of fuel consumption and the rotational speed of the engine 102 is 1000 cubic millimeters per minute and 500 RPM, respectively, then the processing components 122 may employ a multiplier value of 1.75 in calculating the rate of degradation of oil. However, if the rate of fuel consumption and the rotational speed of the engine 102 is 950 cubic millimeters per minute and 2500 RPM, respectively, then the processing components 122 may employ a multiplier value of 1.2 in calculating the rate of degradation of oil. Therefore, the step of calculating the rate of degradation of oil based on the received engine variable 106 and the determined operating parameter 114 includes generating one or more maps to correlate the operating parameters 114 while taking into account the determined engine variables 106.

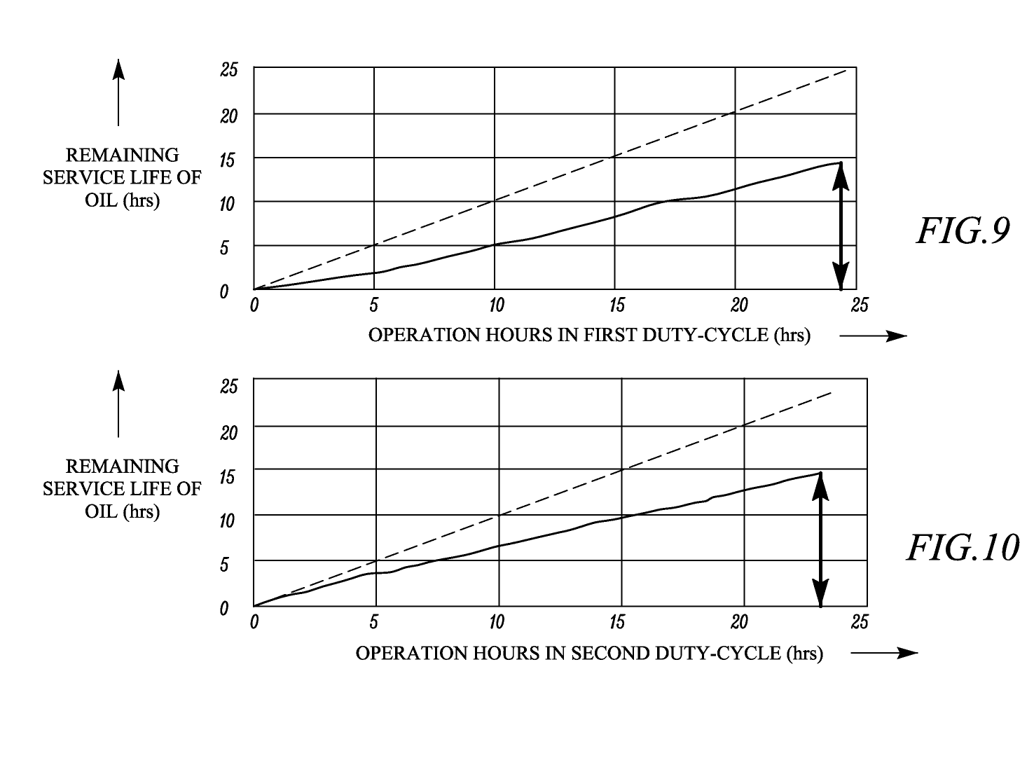

Referring back to FIG. 2, at step 208, the method 200 further includes determining a remaining service-life of the oil from the calculated rate of degradation. Referring to FIGS. 9-10, exemplary plots of degradation in oil condition is shown. Specifically, FIG. 9 is a plot obtained from the first duty-cycle of the engine 102, for example, on the first day of operation of the engine 102, while FIG. 10 is a plot obtained from the second duty-cycle of the engine 102, for example, on the second day of operation of the engine 102. Further, a dashed line is used to represent an empirically calculated degradation in the oil, for example, a service life estimated by an oil manufacturer under test conditions, while a solid line is used to represent an actual degradation in the oil calculated by using the method 200 of the present disclosure.

Referring to FIGS. 9-10, the actual degradation during both duty cycles i.e. the first duty-cycle and the second duty-cycle of the engine 102 is shown to be lesser than the empirically calculated degradation of the oil for the respective duty-cycles. Further, the plots show different rates of degradation in the oil for the first duty-cycle and the second duty cycle. The rates of degradation may be understood to be different since a slope exhibited by the solid line of FIG. 9 is different from a slope exhibited by the solid line of FIG. 10.

In the exemplary representation of FIG. 9, it is shown that after a 15 hour first duty-cycle, the degradation in service life of the oil is approximately 8 hours of oil life. However, in the exemplary representation of FIG. 10, it is shown that after a 15 hour second duty-cycle, the amount of degradation in the oil is approximately 10 hours of oil life. Therefore, it can be inferred that the oil has undergone increased degradation during the second-duty cycle when compared to the degradation in the first-duty cycle. Further, a difference between the dashed lines and the solid lines of the respective duty-cycles shown in FIGS. 9 and 10 may indicate that the engine variables 106 and the operating parameters 114 of the engine 102 affect the rate of degradation of oil and hence, affect a service life of the oil.

Although in FIGS. 9-10, the actual degradation is shown to be lesser than the empirically calculated degradation of the oil during the first duty-cycle and the second-duty cycle, a person having ordinary skill in the art will acknowledge that the reverse is also possible, i.e. the actual degradation in oil may be more than the empirically calculated degradation for a given duty-cycle, when the solid line has an increased slope as compared to a slope of the dashed line.

Using the actual degradation in the oil from the solid lines in the plots of FIGS. 9-10, in one exemplary embodiment, the remaining service life of oil may be calculated by subtracting the actual degradation of oil from the service life estimated by an oil manufacturer. The remaining service life of oil may be determined from an exemplary mathematical equation:

R_{SL}=T−T₁ Equation (1);

wherein R_{SL} is the remaining service life of oil (R_{SL}), T is the service life estimated by manufacturer, and T1 is the actual degradation of oil. The terms R_{SL}, T, and T₁ may be expressed in terms of hours (hrs).

Although equation (1) may be employed to calculate the remaining service life of oil (R_{SL}), it is to be noted that the equation (1) disclosed herein, is merely exemplary in nature and hence, non-limiting of this disclosure. Various constants or co-efficients may be additionally associated to the terms of the equation (1) account for other pre-determined factors while determining the remaining service life of oil (R_{SL}). These pre-determined factors may be associated with the oil and/or the operation of the machine such as, but not limited to, the presence of additives in the engine 102 or oil, the grade of motor oil used etc. Therefore, equation (1) may be suitably modified to determine the remaining service life of oil depending on specific conditions encountered in a given application, and may be mathematically represented by equation (2) as follows:

R_{SL}=(x·T)−(y·T₁) Equation (2);

wherein x may be a constant associated with the empirically estimated oil life while y may be a constant employed to the actual degradation determined from the exemplary plots of FIGS. 9 and 10.

### INDUSTRIAL APPLICABILITY

Manufacturers of oil typically provide an estimate of service-life of oil based on empirical methods of calculation. However, the oil may be used in a wide variety of machines, for example, engines of automobiles, engines of diesel gensets, and engines employed in other industrial applications. Moreover, the types of engines, and the corresponding operating parameters vary from one industrial application to another. For example, an engine in a motor grader may be subject to different conditions than an engine in a heavy mining truck. Moreover, similar engines employed in different applications may also experience different operating conditions. Consequently, the oil within the engines may also be subject to different operating parameters associated with the engines.

The different types of engines, fuels, grades of oil, and operating conditions of the engine pose difficulty to an operator in estimating the actual service life of the oil. In some cases, the operator may replace oil before the service life is over, thereby incurring additional costs associated with premature replacement of oil. In other cases, the oil may expire prematurely, i.e., before the empirically estimated service-life provided by the manufacturer, thereby leaving the engine running in degraded oil. Therefore, in order to avoid detrimental effects to the engine due to running in degraded oil, it may be helpful to monitor and assess the condition of oil from time to time. This periodic assessments of oil condition may help an operator to know the service-life remaining in the oil and enable the operator to take necessary actions or steps associated with replacement of oil.

The replacement of oil in engines is typically scheduled based on factors such as engine mileage, or time elapsed from last change of oil in the engine. However, these factors may be insufficient to determine accurately the current condition or quality of oil and hence, may be insufficient to determine the remaining service life of oil.

The method 200 of the present disclosure is employed to assess the oil condition in the engine 102. The method 200 employs various types of engine variables 106 and operating parameters 114 to assess the oil condition in the engine 102. The method 200 includes generating two-dimensional and three-dimensional maps that help improve accuracy in determining the remaining service-life of the oil. In an embodiment, the system 100 may be operational to perform the steps of the method 200 based on an input from the operator. Alternatively, the system 100 may be pre-programmed to execute the steps of the method 200 at pre-specified time intervals. Optionally, the system 100 may be configured to operate continuously during duty-cycles of the engine 102 and assess the oil condition in the engine 102. Therefore, with use of the present method 200, it is possible to closely monitor the oil quality from time to time and prevent any detrimental effects to the engine 102.

The processing components 122 of the system 100 may embody a single microprocessor or multiple microprocessors for executing the steps of the method 200. Numerous commercially available microprocessors can be configured to perform the functions of the processing components 122. It should be appreciated that the system 100 could readily be embodied in a general microcontroller or directly into the ECM associated with the engine 102 such that the system 100 can readily receive the engine variables 106, and determine many operating parameters 114 of the engine 102. Therefore, the system 100 of the present disclosure may be minimally complex, easy to install and integrate with the engine 102 or the ECM of the engine 102. Moreover, the system 100 may be used on any type of engine 102 employing any type of fuel, or any type of oil thereby rendering the system 100 versatile for measurement in various industrial settings.

Apart from the processing components 122 and the memory unit 110, the system 100 may further include secondary storage devices and other commonly known components for running an application. Further, various circuits may be associated with the system 100, such as power supply circuitry, signal processing circuitry, solenoid driver circuitry, and other types of circuitry. Various routines and/or algorithms can be programmed within the system 100 for execution of the method 200 and for assessment of oil condition in the engine 102.

Although the method 200 of the present disclosure is employed to assess oil condition in the engine 102, the method 200 may be alternatively employed to assess oil condition at other locations of a machine. The method 200 may be adapted to assess oil condition at locations such as, but not limited to, a transmission, brakes, actuation cylinders, or other hydraulic components typically known to employ oil for operation. Accordingly, the method 200 may be suitably modified to receive variables and operating parameters pertaining to the associated locations and/or hydraulic components. A person having ordinary skill in the art will appreciate that the method 200 disclosed herein may be beneficially incorporated at the other locations for monitoring the service life of oil therein.

While aspects of the present disclosure have been particularly shown and described with reference to the embodiments above, it will be understood by those skilled in the art that various additional embodiments may be contemplated by the modification of the disclosed machines, systems, and methods without departing from the spirit and scope of what is disclosed. Such embodiments should be understood to fall within the scope of the present disclosure as determined based upon the claims and any equivalents thereof.

We claim:

1. A method of assessing oil condition in an engine, the method comprising:
receiving an engine variable comprising at least one of: a type of the engine, a displacement of the engine, a property of fuel used in the engine, and a property of oil used in the engine;
determining an operating parameter of the engine, the operating parameter comprising at least one of: an oil temperature, an oil pressure, a duty-cycle of the engine, a load on the engine, a rotational speed of the engine, a power output of the engine, a rate of fuel consumption by the engine, and a time period elapsed since last change of oil in the engine;
calculating a rate of degradation of oil based on the determined engine variable and the determined operating parameter; and
determining a remaining service-life of the oil from the calculated rate of degradation.
